(19) United States
(12) Patent Application Publication
(10) Pub. No.: US 2006/0153857 A1
(43) Pub. Date: Jul. 13, 2006

(54) Method of treating staphylococcus aureus infection

(75) Inventors: Gary Horwith, Gaithersburg, MD (US); Ali Ibrahim Fattom, Rockville, MD (US)

Correspondence Address: FOLEY AND LARDNER LLP;SUITE 500, 3000 K STREET NW, WASHINGTON, DC, 20007, US

(73) Assignee: NABI BIOPHARMACEUTICALS

(21) Appl. No.: 11/245,430

(22) Filed: Oct. 7, 2005

Related U.S. Application Data
(60) Provisional application No. 60/642,093, filed on Jan. 10, 2005.

(51) Int. Cl. A61K 39/40; A61K 35/54

(52) U.S. Cl. 424/165.1; 424/581

(57) ABSTRACT

The present invention provides a method of preventing or treating bacteremia caused by Staphylococcus aureus, comprising administering a monoclonal or polyclonal antibody composition comprising antibodies specific for one or more S. aureus antigens. In one specific embodiment, the composition is a hyperimmune specific IGIV composition. In another specific embodiment, the composition comprise antibodies to a capsular polysaccharide S. aureus antigen, such as the Type 5 and/or Type 8 antigens. In another embodiment, the composition comprises monoclonal antibodies to a capsular polysaccharide S. aureus antigen. This method provides an effective tool for preventing or treating S. aureus bacteremia, and can be used alone or in combination with other therapies.

### CROSS-REFERENCE TO RELATED PATENT APPLICATIONS

This application claims benefit of U.S. patent application No. 60/642,093, filed Jan. 10, 2005, which is incorporated in its entirety herein by reference.

### BACKGROUND OF THE INVENTION

Staphylococcus aureus infections represent a significant cause of illness and death, accounting for about 20% of all cases of bacteremia. Staphylococcus aureus bacteria are the most common cause of hospital-acquired infections and are becoming increasingly resistant to antibiotics. An estimated 12 million patients are at risk for developing a S. aureus infection each year in the U.S. alone. Within the country's 7,000 acute care hospitals, S. aureus is the leading cause of hospital-acquired bloodstream infections and is becoming increasingly resistant to antibiotics, rendering the infections potent causes of illness and death with a crude mortality rate of about 25%. A study by Ruben et al., EMERG. INFECT. DIS. 5:9-17 (1999), showed that the average hospital stay for subjects with a Staphylococcus aureus infection is 20 days, which is nearly three times the average stay for any other type of hospitalization, and the average cost per case is $32,000. Thus, Staphylococcus aureus infection is a major public health concern.

Staphylococcus aureus bacteria, often referred to as “staph,” “Staph. aureus,” or “S. aureus,” are commonly carried on the skin or in the nose of healthy individuals. Approximately 20-30% of the population is colonized with S. aureus at any given time. These bacteria often cause minor infections, such as pimples and boils. However, S. aureus also causes serious and potentially deadly bacteremia, which is a medical condition characterized by viable bacteria present in the blood stream.

The individuals most at risk for bacteremia include newborns, nursing mothers, surgical patients, individuals with foreign bodies (i.e., invasive devices such as, e.g., catheters, prostheses, artificial hips, knees or limbs, dialysis access grafts, pacemakers and implantable defilibrators), immunocompromised patients, such as chemotherapy patients and patients taking immunosuppressant drugs (e.g. transplant patients, cancer patients and HIV positive individuals), patients with chronic illnesses, and patients being cared for in hospitals, nursing homes, dialysis centers or similar institutions. Patients who have been treated for a serious staph infection and released from the hospital also may be at a very high risk for a recurrence of another serious staph infection within a relatively short period of time. See CLINICAL INFECTIOUS DISEASES 2003; 36: 281-285. In at-risk subjects, and sometimes even in otherwise healthy individuals, S. aureus caused bacteremia can cause systemic manifestations and inflammation.

Common symptoms of bacteremia include tachypnea, chills, elevated temperature, abdominal pain, nausea, vomiting, and diarrhea. Often, patients with bacteremia initially present with warm skin and diminished mental alertness. A drop in blood pressure, i.e. hypotension, may also be present, indicating the start of sepsis. Sepsis generally refers to a systemic infection, such as a case of S. aureus caused bacteremia that causes systemic manifestations of inflammation. A systemic inflammatory response is defined by THE MERCK MANUAL OF DIAGNOSIS AND THERAPY § 13, Ch. 156, 100^{th} Ed. (Beers & Berkow eds. 2004), as the presence of at least two of the following objective measurements: (1) temperature greater than 38° C. or less than 36° C.; (2) heart rate greater than 90 beats/min.; (3) respiratory rate greater than 20 breaths/min or PaCO₂ less than 32 mm Hg; and (4) WBC count greater than 12,000 or less than 4000 cells/μL, or greater than 10% immature forms. In some cases, bacteremia can result in septic shock and ultimately death.

Bacteremia caused by S. aureus can sometimes be treated successfully using antibiotics. However, even with a number of antibiotics available today, S. aureus infections are still associated with significant patient mortality. Bacteremia has an estimated mortality rate ranging from 16% to 43%. Left-sided endocarditis in persons who do not use injection drugs is associated with an estimated patient mortality of 20% to 40%. Vertebral osteomyelitis is associated with a reported mortality of 16%.

Bacteremia can progress rapidly, leaving little time for conventional antibiotics to work. Patients may initially present with relatively benign symptoms, such as fever and chills. However, these symptoms can rapidly worsen to include hypotension, a hallmark of septicemia. By the time a diagnosis is made, the condition may have progressed too far to treat effectively with known methodologies.

In some patients, conventional antibiotic treatment is complicated by patient allergies to antibiotics. For example, patients may be allergic to one or more of the preferred antibiotics used to treat S. aureus infections. The allergic reaction can vary from minor gastrointestinal problems to anaphylaxis. This situation can be further complicated in instances where the S. aureus is resistant to one or more antibiotics. Thus, care providers can be forced to choose between risking a potentially serious allergic reaction and relying on an inferior therapeutic agent (such as a non-preferred antibiotic) to curtail a potentially deadly systemic infection.

Another problem is that S. aureus bacteria are becoming increasingly resistant to available antibiotics. For example, methicillin resistant S. aureus (MRSA) has become a common cause of S. aureus caused bacteremia. Worldwide it is estimated that over 95% of patients with S. aureus infections no longer respond to first-line antibiotics, such as penicillin or ampicillin. Methicillin is an alternative treatment, but over 57% of strains of S. aureus are now Methicillin-resistant (MRSA) in the United States. For example, in 1999, 54.5% of all S. aureus isolates reported in the National Noscomial Infections Surveillance System (NNISS) were methicillin resistant. The Centers for Disease Control estimate that in 2002 there were approximately 100,000 cases of hospital-acquired MRSA infections in the United States and the problem of these infections is only worsening. The rates of Methicillin-resistance are even greater in certain Asian and European countries, (e.g., 72% MRSA rate in Japan; 74% in Hong Kong). While vancomycin usage is considered a last line of defense for treating S. aureus infections, vancomycin intermediate strains (VISA) and vancomycin resistant strains (VRSA) are becoming increasingly common. These antibiotic resistant strains currently cause problems in treating bacteremia caused by S. aureus, and these problems will only become worse unless new treatment tools are developed.

Thus, antibiotic therapy of S. aureus bacteremia is sometimes inadequate. This may be particularly true for patients with compromised immune systems. For example, antibiotic therapy alone may not effectively treat bacteremia in patients recovering from surgery and/or taking immunosuppressant drugs. Newborns are also difficult to treat due to their immature immune systems. These patients sometimes lack the strength to overcome a systemic infection despite aggressive antibiotic therapy.

Hyperimmune specific intravenous immunoglobulin (IGIV) compositions comprising antibodies specific for S. aureus have been investigated and used in the prevention of S. aureus infection. For example, Altastaph™ (comprising antibodies to S. aureus Type 5 and Type 8 antigens) has been used to provide immediate protection against S. aureus infections in low birth-weight infants, and is being investigated to provide short-term, immediate protection, to patients who either cannot wait for a vaccine effect to occur or whose immune system is too compromised to mount an adequate response to a vaccine. However, such IGIV compositions heretofore have not been demonstrated to be effective in treating existing S. aureus infection.

Thus, there is a need for new methods of preventing and treating bacteremia caused by S. aureus, including methods for preventing and treating bacteremia caused by antibiotic resistant strains of S. aureus.

### SUMMARY OF THE INVENTION

The present invention relates to methods for preventing and treating bacteremia caused by S. aureus using an antibody composition comprising monoclonal or polyclonal antibodies specific for S. aureus.

In one embodiment, the present invention provides a method of preventing or treating S. aureus bacteremia, comprising administering to a patient at risk of or suffering from S. aureus bacteremia an effective amount of a monoclonal or polyclonal antibody composition comprising antibodies specific for one or more antigens of Staphylococcus aureus.

In one specific embodiment, the antibody composition is a polyclonal antibody composition, and is an IGIV composition. In another specific embodiment, the polyclonal antibody composition is a hyperimmune specific IGIV composition. In another specific embodiment, the polyclonal antibody composition comprises recombinant polyclonal antibodies.

In another specific embodiment the antibody composition is a monoclonal antibody composition that comprises monoclonal antibodies specific for one or more antigens of Staphylococcus aureus.

In accordance with one aspect of the invention, the monoclonal or polyclonal antibody composition comprises antibodies specific to one or more capsular polysaccharide antigens of Staphylococcus aureus, such as antibodies specific to one or more antigens selected from the group consisting of the Type 5 antigen, the Type 8 antigen, and the 336 antigen. Compositions comprising antibodies specific to two or more such antigens are specifically contemplated.

In accordance with another aspect of the invention, the bacteremia is characterized by a persistent fever. Additionally or alternatively, the bacteremia is caused by an antibiotic resistant Staphylococcus aureus, such as Staphylococcus aureus resistant to methicillin and/or vancomycin.

In accordance with another aspect of the invention, the patient is immunocompromised. Additionally or alternatively, the patient is allergic to at least one antibiotic used to treat Staphylococcus aureus.

In accordance with another aspect of the invention, the method further comprises an additional therapy against Staphylococcus aureus infection, such as a therapy comprising the administration of one or more antibiotic or antimicrobial agents, such as lysostaphin.

### DETAILED DESCRIPTION

The present invention provides a method of preventing or treating bacteremia caused by S. aureus, comprising administering an antibody composition comprising monoclonal or polyclonal antibodies specific for S. aureus. In a particular embodiment, the antibody composition is a polyclonal antibody composition such as an intravenous immunoglobulin (IGIV) composition comprising antibodies specific for one or more S. aureus antigens. For example, the polyclonal antibody composition may be a hyperimmune specific IGIV composition specific for one or more S. aureus antigens. Alternatively, the polyclonal antibody composition may comprise recombinantly produced polyclonal antibodies against S. aureus. In another specific embodiment, the polyclonal antibody composition comprises opsonizing antibodies.

In another particular embodiment, the antibody composition comprises monoclonal antibodies specific for one or more S. aureus antigens. The composition may comprise recombinantly produced monoclonal antibodies. In another specific embodiment, the monoclonal antibody composition comprises opsonizing antibodies.

The inventive method provides an effective tool for preventing or treating S. aureus bacteremia, and can be used alone or in combination with other therapies, such as antibiotic therapies or therapies using other agents, such as antimicrobial agents, bacteriocidal agents and bacteriostatic agents. The method is effective against antibiotic-resistant strains of S. aureus and, because the method does not require the use of antibiotics, is useful for patients who are allergic to one or more of the antibiotics used to treat S. aureus infection.

The following detailed description of the invention illustrates certain exemplary embodiments and allows a better understanding of the claimed invention.

Unless otherwise specified, “a”, “an”, and “the” as used herein mean “one or more.”

As used herein, the term “antibody” includes monoclonal and polyclonal antibodies, whole antibodies, antibody fragments, and antibody subfragments that exhibit specific binding to a specific antigen of interest. Thus, “antibodies” can be whole immunoglobulin of any class, e.g., IgG, IgM, IgA, IgD, IgE, chimeric antibodies or hybrid antibodies with dual or multiple antigen or epitope specificities, or fragments, e.g., F(ab′)₂, Fab′, Fab and the like, including hybrid fragments, and additionally includes any immunoglobulin or any natural, synthetic or genetically engineered protein that acts like an antibody by binding to a specific antigen to form a complex. For example, Fab molecules can be expressed and assembled in a genetically transformed host like E. coli. A lambda vector system is available thus to express a population of Fab's with a potential diversity equal to or exceeding that of subject generating the predecessor antibody. See Huse, W. D., et al., Science 246: 1275-81 (1989). Such Fab's are included in the definition of“antibody.” The ability of a given molecule, including an antibody fragment or subfragment, to act like an antibody and specifically bind to a specific antigen can be determined by binding assays known in the art, for example, using the antigen of interest as the binding partner.

As used herein, “bacteremia” means the presence of viable bacteria in the blood of an individual (human or other animal). “Bacteremia caused by S. aureus” or “S. aureus bacteremia” refers to bacteremia in which at least some of the bacteria in the blood are S. aureus. Other species of bacteria also may be present.

As used herein, “intravenous immunoglobulin (IGIV)” means an immunoglobulin composition suitable for intravenous administration. The IGIV composition can be administered by a number of routes, including intravenously, intramuscularly and subcutaneously. “Specific IGIV” refers to IGIV specific for one or more specified antigens. The one or more antigens can be any antigen of interest, such as an antigen characteristic of a pathogenic organism, such as S. aureus.

“Hyperimmune specific IGIV” refers to an IGIV preparation obtained by purifying immunoglobulin from an individual who has been challenged with one or more specified antigens, such as an individual who has been administered a vaccine comprising one or more antigens of interest. The purified immunoglobulin comprises antibodies specific to the specific antigen(s) of interest. The individual from whom the immunoglobulin is obtained can be a human or other animal.

As used herein, “recombinantly produced polyclonal antibodies” means polyclonal antibodies produced by recombinant methods, such as methods analogous to those described in U.S. Patent Application 2002/0009453 (Haurum et al.).

As used herein, “recombinantly produced monoclonal antibody” means monoclonal antibodies produced by recombinant methods, such as those well known in the art.

As used herein, “opsonizing antibodies” means antibodies that attach to the invading microorganism (i.e., S. aureus) and other antigens to make them more susceptible to the action of phagocytes.

In accordance with the present invention, bacteremia is prevented or treated by a method comprising administering to the infected patient (human or other animal) a monoclonal or polyclonal antibody composition comprising antibodies specific for S. aureus.

In a particular embodiment, the composition is a polyclonal antibody composition which is an intravenous immunoglobulin preparation (IGIV) comprising antibodies specific for one or more S. aureus antigens, such as the Type 5 antigen, the Type 8 antigen and/or the 336 antigen. The polyclonal antibody composition may be a hyperimmune specific IGIV composition. Alternatively, the polyclonal antibody composition may comprise antibodies obtained by other means, such as recombinantly produced polyclonal antibodies. In another specific embodiment, the polyclonal antibody composition comprises opsonizing antibodies.

In another specific embodiment, the antibody composition is a monoclonal antibody composition that comprises monoclonal antibodies specific for S. aureus. The antibody composition may comprise monoclonal antibodies specific for one or more S. aureus antigens, such as the Type 5 antigen, the Type 8 antigen and/or the 336 antigen. The monoclonal antibodies may be obtained by conventional hybridoma technology or they may be obtained by other means, such as by recombinant methods known in the art. In one specific embodiment, the monoclonal antibody composition comprises opsonizing antibodies.

Bacteremia is most common in certain risk categories, although it can occur in anyone. As discussed above, these risk categories include newborns, nursing mothers, surgical patients, individuals with foreign bodies (i.e., invasive devices such as, e.g., catheters, prostheses, artificial hips, knees or limbs, dialysis access grafts, pacemakers and implantable defilibrators), immunocompromised patients, such as chemotherapy patients and patients taking immunosuppressant drugs (e.g. transplant patients, cancer patients and HIV positive individuals), patients with chronic illnesses, and patients being cared for in hospitals, nursing homes, dialysis centers or similar institutions. Patients who have been treated for a serious staph infection and released from the hospital also may be at a very high risk for a recurrence of another serious staph infection within a relatively short period of time. The use of the present invention to prevent or treat bactermia in patients with weak immune systems, such as patients in one or more of these risk categories, can be particularly advantageous. For example, in immunocompromised patients and newborns, a monoclonal or polyclonal antibody composition (such as a hyperimmune specific IGIV) specific for one or more S. aureus antigens may boost the effectiveness of the patient's own immune system, improving the odds of successful treatment.

Any type of bacteremia caused by S. aureus can be prevented or treated using the present invention. As defined above, the phrases “bacteremia caused by S. aureus” and “S. aureus bacteremia” refer to bacteremia in which at least some of the bacteria in the blood are S. aureus, even if other species of bacteria are present. The bacteremia prevented or treated in accordance with the present invention can be caused by any strain of S. aureus, including antibiotic resistant strains of S. aureus. Common antibiotic resistant strains include methicillin resistant strains (MRSA) and vancomycin resistant strains (VISA and VRSA). The S. aureus prevented or treated by the present invention also can be a strain that is resistant to more than one antibiotic. Additionally, the bacteremia prevented or treated by the present invention can be caused by more than one strain of S. aureus, including one, two, three, or more strains of S. aureus. Also, the bacteremia can be a persistent bacteremia.

The bacteremia prevented or treated in accordance with the present invention also can involve bacteria other than S. aureus. In other words, bacteria other than S. aureus can be present in the patient's blood. For example, other bacteria such as Gram negative or Gram positive bacteria may be present. Examples of other bacteria associated with bacteremia include, but are not limited to, Staphylococcus sp., Streptococcus sp., Pseudomonas sp., Haemophilus sp., Enterococcus sp., and Esherichia coli. The present invention is effective to prevent or treat S. aureus infection regardless of the presence of other bacteria.

In one embodiment, the monoclonal or polyclonal antibody composition used in the present invention comprises monoclonal or polyclonal antibodies specific to at least one S. aureus antigen. For example, the composition can comprise antibodies to capsular polysaccharide antigens, such as the Type 5 and Type 8 antigens described in Fattom et al., INF. AND IMM. 58:2367-2374 (1990), and Fattom et al., INF. AND IMM. 64:1659-1665 (1996). Additionally or alternatively, the composition may comprise antibodies specific to the 336 antigen described in U.S. Pat. No. 6,537,559 to Fattom et al. Other S. aureus antigens are known in the art, see Adams et al., J. CLIN. MICROBIOL. 26(6):1175-80 (1988), Rieneck et al., BIOCHIM. BIOPHYS. ACTA. 1350(2):128-32 (1997), and O'Riordan et al., CLIN. MICROBIOL. REV. 17(1):218-34 (2004), and compositions comprising polyclonal antibodies specific to those antigens are useful in the present invention.

Additionally or alternatively, the antibody composition also may comprise antibodies specific for other pathogens, including antibodies specific for other Staphylococcal antigens, such as antibodies specific for S. epidermis antigens, such as the PS1 and GP1 antigens. PS1 is a S. epidermidis Type II antigen, and is described, for example, in U.S. Pat. Nos. 5,961,975 and No. 5,866,140. PS1 is an acidic polysaccharide antigen that can be obtained by a process that comprises growing cells of an isolate of S. epidermidis that agglutinates antisera to ATCC 55254 (a Type II isolate). The GP1 antigen is described in published U.S. patent application 2005/0118190, now U.S. Pat. No. 6,936,258. GP1 is common to many coagulase-negtive strains of Staphylococcus, including Staphylococcus epidermis, Staphylococcus haemolyticus, and Staphylococcus hominis. The antigen can be obtained from the strain of Staphylococcus epidermis deposited as ATCC 202176.

Another Staphylococcus antigen of interest is described in WO 00/56357 and comprises amino acids and a N-acetylated hexosamine in an α configuration, contains no O-acetyl groups, and contains no hexose. It specifically binds with antibodies to a Staphylococcus strain deposited under ATCC 202176. Amino acid analysis of the antigen shows the presence of serine, alanine, aspartic acid/asparagine, valine, and threonine in molar ratios of approximately 39:25:16:10:7. Amino acids constitute about 32% by weight of the antigen molecule. Antibodies specific to this antigen can be included in the antibody composition of the present invention.

The antibody composition also may comprise antibodies specific for other bacteria, such as other Gram negative or Gram positive bacteria. For example, the antibody composition may comprise antibodies specific for Streptococcus sp., Pseudomonas sp., Haemophilus sp., Enterococcus sp., and Esherichia coli. Antigen-based vaccines against infection by these bacteria are known in the art, and can be used to raise antibodies for use in the invention. For example, any known Streptococcal vaccine can be used to raise antibodies specific for Streptococcus sp. Likewise, the E. coli lipopolysaccharide antigen (LPS) can be used to raise antibodies specific for E. coli, and capsular polysaccharide antigens of Pseudomonas sp. and Haemophilus sp. can be used to raise antibodies specific for those bacteria. Antigens of Enterococcus sp. are described, for example, in U.S. Pat. No. 6,756,361, and can be used to raised antibodies specific for those bacteria.

The antibodies can be specific for a native form of the antigen, can be specific for a modified form of the antigen, or can be specifically recognize both native and modified forms of the antigen. For example, native forms of both the Type 5 and Type 8 antigens comprise a polysaccharide backbone bearing O-acetyl groups. Antibodies specific for the O-acetylated forms of these antigens are useful in the present invention. The O-acetyl groups can be removed, for example, by treating the antigen with a base or subjecting the antigen to basic pH. Antibodies specific for the de-O-acetylated forms of these antigens also are useful in the present invention. Moreover, antibodies that specifically recognize both the O-acetylated and the de-O-acetylated forms of these antigens are useful in the present invention.

In one embodiment, the monoclonal or polyclonal antibody composition comprises antibodies specific to both the Type 5 and Type 8 antigens. In another embodiment, the composition comprises antibodies specific to the 336 antigen. In yet another embodiment, the composition comprises antibodies specific to the Type 5, Type 8 and 336 antigens. At least one of the Type 5 antigen, Type 8 antigen, or the 336 antigen are present in nearly every case of S. aureus caused bacteremia. Thus, monoclonal or polyclonal antibody compositions comprising antibodies specific to one or more of those antigens can be used in accordance with the present invention to prevent or treat S. aureus bacteremia.

Other monoclonal or polyclonal antibody compositions useful in the present invention will be readily apparent to those skilled in the art and can be prepared by methods analogous to those described in more detail below.

The present invention contemplates the use of a single polyclonal antibody composition comprising antibodies against one or more S. aureus antigens, such as the Type 5, Type 8 and 336 antigens, and also contemplates the use of a plurality of polyclonal antibody compositions, each comprising antibodies against one or more S. aureus antigens or antibodies against at least one S. aureus antigen and antibodies against at least one other pathogen, such as antibodies against at least one S. epidermis antigen. If a plurality of compositions are used, they may be combined prior to administration, or they may be administered separately, at the same time or at different times.

The present invention also contemplates the use of a single monoclonal antibody composition comprising antibodies against one or more S. aureus antigens, such as the Type 5, Type 8 and 336 antigens. Such a composition may be referred to as an “engineered oligoclonal” composition, and may comprise, for example, a mixture of monoclonal antibodies to one or more of the S. aureus Type 5, Type 8, and 336 antigens. The invention also contemplates the use of a plurality of monoclonal antibody compositions, each comprising antibodies against one or more S. aureus antigens. If a plurality of compositions are used, they may be combined prior to administration, or they may be administered separately, at the same time or at different times.

The present invention also contemplates the use of two or more antibody compositions, at least one of which is a monoclonal antibody composition and at least one of which is a polyclonal antibody composition. In this embodiment, the antibody compositions may be combined prior to administration, or they may be administered separately, at the same time or at different times.

When mixtures of antibodies are used, the antibodies can be linked together chemically to form a single polyspecific molecule capable of binding to two or more antigens of interest. One way of effecting such a linkage is to make bivalent F(ab′)₂ hybrid fragments by mixing two different F(ab′)₂ fragments produced, e.g., by pepsin digestion of two different antibodies, reductive cleavage to form a mixture of Fab′ fragments, followed by oxidative reformation of the disulfide linkages to produce a mixture of F(ab′)₂ fragments including hybrid fragments containing a Fab′ portion specific to each of the original antigens. Methods of preparing such hybrid antibody fragments are disclosed in Feteanu, LABELED ANTIBODIES IN BIOLOGY AND MEDICINE 321-23, McGraw-Hill Int'l Book Co. (1978); Nisonoff, et al., Arch Biochem. Biophys. 93: 470 (1961); and Hammerling, et al., J. Exp. Med. 128: 1461 (1968); and in U.S. Pat. No. 4,331,647.

Other methods are known in the art to make bivalent fragments that are entirely heterospecific, e.g., use of bifunctional linkers to join cleaved fragments. Recombinant molecules are known that incorporate the light and heavy chains of an antibody, e.g., according to the method of Boss et al., U.S. Pat. No. 4,816,397. Analogous methods of producing recombinant or synthetic binding molecules having the characteristics of antibodies are included in the present invention. More than two different monospecific antibodies or antibody fragments can be linked using various linkers known in the art.

In accordance with the present invention, the antibody profile of the monoclonal or polyclonal antibody composition can be selected depending on the particular antigen profile of the infection being treated. In the alternative, a broad-spectrum composition, such as one containing antibodies specific to two or more S. aureus antigens or one containing antibodies specific to at least one S. aureus antigen and at least one other pathogen, such as at least one S. epidermis antigen, can be administered without the need to determine the antigen profile of the targeted infection. A combination therapy approach, i.e., a method using a monoclonal or polyclonal antibody composition comprising antibodies specific to two or more antigens, may prove to be particularly useful in patients afflicted with life-threatening infections, such as patients suffering from persistent and/or antibiotic resistant bacteremia.

As noted above, in one embodiment, the composition is a hyperimmune specific IGIV composition. The hyperimmune specific IGIV composition can be prepared using methods well known in the art. Typically, hyperimmune specific IGIV is obtained by administering to a subject a composition, such as a vaccine, comprising the specific antigen or antigens of interest. Plasma is harvested from the subject, and the specific immunoglobulin is obtained from the plasma via conventional plasma-fractionation methodology. The subject can be either a human or animal.

Suitable IVIG compositions also can be obtained by screening plasma obtained from a subject that has not been administered a S. aureus antigen (i.e., an unstimulated subject). In this embodiment, plasma from unstimulated subjects is screened for high titers of antibodies to a S. aureus antigen, such as a Type 5, Type 8, or 336 antigen. In accordance with one embodiment, plasma is screened for antibody titers that are 2-fold or more higher than the levels typically found in standard IVIG preparations. The hyperimmune specific IGIV useful in the present invention can contain antibodies specific for any S. aureus antigen(s). For example, the hyperimmune specific IGIV can comprise antibodies to the Type 5, Type 8 and/or 336 antigens discussed above. Those antigens can be used to prepare a hyperimmune specific IGIV following the general procedures outlined above. Additionally or alternatively, the hyperimmune specific IGIV composition can comprise antibodies to other S. aureus antigens, and may also include antibodies to other pathogens, including antibodies to other staphylococcal antigens, such as those referenced above. Those antibodies can be used to prepare hyperimmune specific IGIV for use in the present invention the general procedures outlined above.

StaphVAX® (Nabi® Biopharmaceuticals, Rockville, Md.) is an example of a vaccine that can be used to prepare S. aureus hyperimmune specific IGIV for use in the present invention. StaphVAX® (in development for providing protection in at-risk patients against S. aureus infections) comprises capsular polysaccharide S. aureus Type 5 and Type 8 antigens and stimulates production of antibodies specific to the Types 5 and Type 8 antigens. Hyperimmune specific IGIV specific for Type 5 and Type S. aureus antigens can be obtained from subjects who have been administered this vaccine, and can be used in accordance with the present invention to treat bacteremia caused by S. aureus.

Hyperimmune specific IGIV useful in the present invention also can be prepared using other compositions and vaccines comprising S. aureus antigens that are known or that can be readily developed by one of ordinary skill in the art. For example, U.S. Pat. No. 6,537,559 to Fattom et al. describes a S. aureus vaccine comprising the 336 antigen. Hyperimmune specific IGIV comprising antibodies specific for the S. aureus 336 antigen can be obtained from subjects who have been administered that vaccine,

AltaStaph™ (Nabi® Biopharmaceuticals, Rockville, Md.) is an example of a S. aureus hyperimmune specific IGIV composition useful in the present invention. AltaStaph™ contains high levels of antibodies to the capsular polysaccharide Type 5 and Type 8 antigens from S. aureus. AltaStaph™ is produced by immunizing healthy human volunteers with StaphVAX®. As presently produced, AltaStaph™ is a sterile, injectable 5% solution of human plasma protein at pH 6.2 in 0.075 sodium chloride, 0.15 M glycine and 0.01% polysorbate 80. Each 1 mL of solution contains 50 mg protein, of which greater than 96% is IgG immunoglobulin. IgA and IgM classes are present at concentrations of <1.0 g/L. Approximately 85%o of all S. aureus infections are caused by S. aureus associated with the Type 5 or 8 antigens. Thus, a hyperimmune specific IGIV comprising antibodies specific to the Type 5 and Type 8 antigens, such as AltaStaph™, can be used to effectively treat over 85% of S. aureus infections.

A hyperimmune specific IGIV composition comprising antibodies specific to the Type 5 and Type 8 antigens, such as AltaStaph™, can be used in the present invention alone or in combination with other compositions comprising antibodies specific for one or more S. aureus antigens. For example, another composition comprising antibodies specific for the 336 antigen can be administered to a patient along with the Type 5/Type 8-specific composition. Such administration can be effected by combining the compositions prior to administration, or by administering the compositions separately, at the same time or at different times. The polyclonal antibody composition may comprise recombinantly produced polyclonal antibodies. For example, recombinant polyclonal antibodies specific to S. aureus can be produced by methods analogous to those described in U.S. Patent Application 2002/0009453 (Haurum et al.), using one or more S. aureus antigens as the immunogen.

In accordance with another embodiment, the antibody composition comprises monoclonal antibodies. Suitable monoclonal antibodies can be prepared using conventional hybridoma technology, as outlined below, or by recombinant methods known in the art, such as those described in U.S. Pat. No. 4,816,397.

To form monoclonal antibodies by hybridoma technology, a myeloma or other self-perpetuating cell line is fused with lymphocytes obtained from peripheral blood, lymph nodes or the spleen of a mammal hyperimmunized with the S. aureus antigen of interest. Usually, the myeloma cell line is from the same species as the lymphocytes. Splenocytes are typically fused with myeloma cells using polyethylene glycol 1500. Fused hybrids are selected by their sensitivity to HAT. Hybridomas secreting antibodies specific to the antigen of interest can be identified using an ELISA.

A Balb/C mouse spleen, human peripheral blood, lymph nodes or splenocytes usually are used in preparing murine or human hybridomas. Suitable mouse myelomas for use in the present invention include the hypoxanthine-aminopterin-thymidine-sensitive (HAT) cell lines, such as P3X63-Ag8.653. A typical fusion partner for human monoclonal antibody production is SHM-D33, a heteromyeloma available from the ATCC under the designation CRL 1668.

Monoclonal antibodies can be produced by initiating a monoclonal hybridoma culture comprising a nutrient medium containing a hybridoma that secretes antibody molecules of the appropriate specificity. The culture is maintained under conditions and for a time period sufficient for the hybridoma to secrete the antibody molecules into the medium. The antibody-containing medium is then collected. The antibody molecules then can be isolated further by well known techniques.

Media useful for the preparation of monoclonal antibodies are both well known in the art and commercially available, and include synthetic culture media, inbred mice and the like. An exemplary synthetic medium is Dulbecco's Minimal essential medium supplemented with 20% fetal calf serum. An exemplary inbred mouse strain is the Balb/c.

Other methods of preparing monoclonal antibodies are also contemplated, such as interspecies fusions. Human lymphocytes obtained from infected individuals can be fused with a human myeloma cell line to produce hybridomas which can be screened for the production of antibodies that recognize the antigen of interest, such as the S. aureus antigen(s). Alternatively, a subject immunized with a vaccine comprising the antigen of interest can serve as a source for antibodies suitably used in an antibody composition within the present invention.

Monoclonal antibodies to the S. aureus Type 5 and Type 8 antigens are known in the art, see, e.g., Nelles et al., Infect. & Immun. 49: 14-18 (1985); Karakawa et al. Infect. & Immun. 56: 1090-95 (1988), as are antibodies to S. epidermis, see, e.g., Timmerman et al., J. Med. Microbiol. 35: 65-71 (1991); Sun et al., Clin. Diag. Lab. Immunol., 12: 93-100 (2005). Monoclonal antibodies to other S. aureus antigens, and to the other bacterial antigens referenced above, can be obtained by analogous methods. Purified monoclonal antibodies can be characterized by bacterial agglutination assays using a collection of clinical isolates.

The composition of the present invention optionally may comprise a pharmaceutically acceptable carrier. A pharmaceutically acceptable carrier is a material that can be used as a vehicle for the composition because the material is inert or otherwise medically acceptable, as well as compatible with the active agent, in the context of administration. A pharmaceutically acceptable carrier can contain conventional passive antibody additives like diluents, adjuvants and other immunostimulants, antioxidants, preservatives and solubilizing agents.

The composition may be provided in any desired dosage form, including dosage forms that may be administered to a human intravenously, intramuscularly, or subcutaneously. As noted above, the IGIV compositions of the present invention may be administered intravenously, intramuscularly, or subcutaneously. The monoclonal antibodies also may be administered intravenously, intramuscularly, or subcutaneously. The composition may be administered in a single dose, or in accordance with a multi-dosing protocol.

The appropriate dosages of the therapeutic composition for use in the present invention can be determined by one of ordinary skill in the art by routine methods. The dosages may depend on a number of factors, such as the severity of infection, the particular therapeutic composition used, the frequency of administration, and patient details (e.g. age, weight, immune condition). In some embodiments using hyperimmune specific IGIV, the dosage will be at least about 50 mg hyperimmune specific IGIV per kg of bodyweight (mg/kg), including at least about 100 mg/kg, at least about 150 mg/kg, at least about 200 mg/kg, at least about 250 mg/kg, at least about 300 mg/kg, at least about 350 mg/kg, at least about 400 mg/kg, at least about 450 mg/kg, at least about 500 mg/kg, or higher.

Dosages for monoclonal antibody compositions typically may be lower, such as 1/10 of the dosage of an IVIG composition, such as at least about 5 mg/kg, at least about 10 mg/kg, at least about 15 mg/kg, at least about 20 mg/kg, at least about 25 mg/kg, at least about 30 mg/kg, at least about 35 mg/kg, at least about 40 mg/kg, at least about 45 mg/kg, at least about 50 mg/kg, or higher. Additionally, lower or higher dosages may be appropriate and effective.

The frequency of dosages and number of dosages also depends on a number of factors, such as the severity of infection and patient immune state. Again, the skilled practitioner can determine an appropriate dosing regimen by routine methods. In some embodiments, the dose can be administered at least about once every other day, including at least about once daily and at least about twice daily. The number of doses needed to effectively treat the bacteremia also can vary depending on the particular circumstances. For example, about one, two, three, four, or more doses of monoclonal antibody composition or hyperimmune specific IGIV may need to be administered to effectively treat the infection. A patient with a weakened immune system or particularly severe infection may require more dosages and/or more frequent dosages.

In one embodiment, AltaStaph™ is administered intravenously at a dose of about 200 mg/kg of bodyweight. In other embodiments, the dosage will be at least about 50 mg/kg, at least about 100 mg/kg, at least about 150 mg/kg, at least about 200 mg/kg, at least about 250 mg/kg, at least about 300 mg/kg, at least about 350 mg/kg, at least about 400 mg/kg, at least about 450 mg/kg, at least about 500 mg/kg, or higher dosages. In some embodiments, only about one or two daily doses are administered. However, additional doses can be administered as needed. In one particular embodiment, two daily doses of about 200 mg/kg are administered. Additionally, lower or higher dosages may be appropriate and effective.

The present invention also contemplates an antibody composition comprising an immunostimlatory compound, such as a β-glucan or GM-CSF. Antibody compositions comprising β-glucan are described, for example, in U.S. Pat. No. 6,355,625. Vaccines comprising GM-CSF as an adjuvant are described, for example, in U.S. Pat. No. 5,679,356. Antibody compositions comprising GM-CSF can be prepared and used analogously. See, e.g., Campell et al., J. Perinatol. 20:225-30 (2000).

The present invention also contemplates the use of the monoclonal or polyclonal antibody composition in conjunction with another therapy, such as antibiotic therapies or therapies using other agents, such as antimicrobial agents, bacteriocidal agents and bacteriostatic agents, such as lysostaphin or other peptides or similar agents. The other therapy may be administered before, during or after the monoclonal or polyclonal antibody composition according to any appropriate regimen which can be determined by the skilled artisan.

For example, an antibiotic effective against a staphylococcal pathogen, such as S. aureus, may be administered together (at the same or different time) with the composition comprising monoclonal or polyclonal antibodies specific to S. aureus. Classes of antibiotics that can be used in accordance with the present invention include all classes used to treat staphylococcal infection, including all classes used to treat S. aureus infection. Specific examples include, but are not limited to, penicillinase-resistant penicillins, cephalosporins, and carbapenems. Specific examples of antibiotics that can be used include, penicillin G, ampicillin, methicillin, oxacillin, nafcillin, cloxacillin, dicloxacillin, cephalothin, cefazolin, cephalexin, cephradine, cefamandole, cefoxitin, imipenem, meropenem, gentamicin, vancomycin, teicoplanin, lincomycin, and clindamycin. Methicillin and vancomycin are common antibiotics for treating S. aureus bacteremia can be used in combination with hyperimmune specific IGIV. The dosages of these antibiotics are well known in the art. THE MERCK MANUAL OF DIAGNOSIS AND THERAPY § 13, Ch. 157, 100^{th} Ed. (Beers & Berkow eds. 2004), describes the treatment of bacteremia using convention antibiotics.

In accordance with the invention, antibiotics used in combination with the monoclonal or polyclonal antibody composition to treat S. aureus bacteremia can be administered at any time, for any duration. For example, the antibiotics can be administered, before, after, and/or simultaneously with the polyclonal antibody composition. In some embodiments, relatively few doses of monoclonal or polyclonal antibody composition are administered, such as one or two doses, and conventional antibiotic therapy is employed, which generally involves multiple doses over a period of days or weeks. Thus, the- antibiotics can be taken one, two, three or more times daily for a period of time, such as for at least 5 days, 10 days, or even 14 or more days, while the monoclonal or polyclonal antibody composition is administered only once or twice. In any event, the different dosages, timing of dosages, and relative amounts of monoclonal or polyclonal antibody composition and antibiotics can be selected and adjusted by one of ordinary skill in the art.

Similar dosage amounts and dosing protocols can be used to prevent bacteremia in accordance with the present invention. For example, in some embodiments using hyperimmune specific IGIV, the dosage will be at least about 50 mg hyperimmune specific IGIV per kg of bodyweight (mg/kg), including at least about 100 mg/kg, at least about 150 mg/kg, at least about 200 mg/kg, at least about 250 mg/kg, at least about 300 mg/kg, at least about 350 mg/kg, at least about 400 mg/kg, at least about 450 mg/kg, at least about 500 mg/kg, or higher. Dosages for monoclonal antibody compositions typically may be lower, such as 1/10 of the dosage of an IVIG composition, such as at least about 5 mg/kg, at least about 10 mg/kg, at least about 15 mg/kg, at least about 20 mg/kg, at least about 25 mg/kg, at least about 30 mg/kg, at least about 35 mg/kg, at least about 40 mg/kg, at least about 45 mg/kg, at least about 50 mg/kg, or higher. Additionally, lower or higher dosages may be appropriate and effective. The frequency of dosages and number of dosages required for prevention may depend on a number of factors, including the patient immune state. A single dose may be effective for prevention, although embodiments comprising subsequent administrations are expressly contemplated.

While not being bound by any particular theory, it is believed that the monoclonal or polyclonal antibody composition used in the present invention boosts the ability of the patient's own immune system to fight infection. In particular, antibodies to S. aureus present in the composition attach to the outer capsule of the bacteria as it circulates in the blood, triggering an immune response and enabling the patient's white blood cells to recognize the bacteria and destroy it before it can contribute to more serious infection. On the other hand, conventional antibiotics and other antimicrobial agents attack the invading bacteria more directly, by killing the bacteria and/or preventing the bacteria from replicating. Thus, the use of the monoclonal or polyclonal antibody composition of the present invention (such as a hyperimmune specific IGIV composition) together with another therapy (such as an antibiotic) counters S. aureus infection through two independent routes, making treatment more effective.

### EXAMPLES

The following examples are meant as illustration only and should not be considered an exhaustive or exclusive description of the invention.

### Example 1

### Specific IGIV Prevents MRSA Staphylococcus aureus Infection in Mice

The ability of hyperimmune specific IGIV (AltaStaph™) to protect against S. aureus infection was investigated using a murine model. Fifteen mice were immunized with AltaStaph™. The AltaStaph™ dosage contained 400 μg of specific antibody (total IgG of 9.6 mg/mouse). As a control, another group of fifteen mice received 9.6 mg of muco-exopolysaccharide (MEP) IGIV containing about 15 fig of Type 5 specific IgG. This low-level amount of Type 5 specific IgG is about the same as found in standard “non-specific” IGIV from commercial sources. A third group of mice received 0.5 ml of buffered saline. In addition, all mice received 0.5 ml of saline intraperitoneally 24 hours prior to challenge. This pre-bacterial challenge treatment was shown to slow the rate of mortality subsequent to challenge by bacterial contact.

Mice were challenged intraperitoneally with three different 2×10⁵ colony forming units (CFUs) of S. aureus in 5% mucin. Two of the S. aureus isolates were of European source (a Type 8 and a Type 5 S. aureus), while the third was from United States (a Type 5 S. aureus). The results are shown below in Table 1.

TABLE 1
Use of IGIV to Prevent MRSA S. aureus Infection in Mice

| Material for | MRSA | Number of Surviving | | |
|---|---|---|---|---|
| Passive | Challenge | Mice/Total Number | | |
| Protection | Isolates | Day 1 | Day 2 | Day 5 |
| AltaStaph ™ | Type 8 | 15/15 | 15/15 | 15/15 |
| MEP-IGIV | Isolate K17654 | 1/15 | 1/15 | 0/15 |
| Placebo | (Germany | 3/15 | 0/15 | 0/15 |
| | 2003) | | | |
| AltaStaph ™ | Type 5 | 15/15 | 15/15 | 15/15 |
| MEP-IGIV | Isolate 12 | 6/15 | 6/15 | 6/15 |
| Placebo | (Germany | 1/15 | 1/15 | 1/15 |
| | 1993) | | | |
| AltaStaph ™ | Type 5 | 37/40 | 37/40 | 36/40 |
| MEP-IGIV | Isolate ST021 | 25/40 | 18/40 | 12/40 |
| Placebo | (USA 1993) | 15/40 | 9/40 | 6/40 |

The protection data at five days after challenge showed that AltaStaph™ was able to protect against diverse S. aureus isolates with 90% -100% efficacy. In contrast, mice in the other groups had a mortality rate of at least 40%. Thus, AltaStaph™ confers significant protection against S. aureus.

### Example 2

### Use of Specific IGIV to Treat Staphylococcus aureus Bacteremia in Humans

The use of hyperimmune specific IGIV to treat S. aureus infection was investigated in a double-blinded, placebo-controlled, randomized trial in 40 patients with persistent S. aureus blood stream infections (bacteremia) designed to evaluate the safety of AltaStaph™ and to measure S. aureus specific antibody levels. Patients were randomly allocated to receive two intravenous doses of AltaStaph™ or saline placebo in combination with standard-of-care treatment, which included treatment with antibiotics. The results of the study demonstrated that AltaStaph™ was well tolerated and no drug-related, serious adverse events were reported. Patients treated with AltaStaph™ were able to maintain antibody titers at or above levels previously estimated to be protective against S. aureus infections in patients with end-stage renal disease (ESRD) by Shinefield et al. N. ENG. J. MED. 14: 491-96 (2002). In addition, as outlined below, AltaStaph™ treatment was associated with a substantial reduction in time to hospital discharge.

The human subjects in the trial had documented S. aureus bacteremia with fever. S. aureus bacteremia with fever was defined as a positive S. aureus blood culture and a temperature of at least 38° C. occurring at least 24 hours after the positive blood culture.

Subjects meeting the requirements were administered two doses of 200 mg per kg of bodyweight (mg/kg) of AltaStaph™ approximately 24 hours apart. Before administration, the AltaStaph™ was placed into either a 500 mL or 1 L sterile IV bag or glass bottle without any dilution. (20 ml AltaStaph™ contains 1000 mg IVIG.) The placebo group received 4 mL/kg of 0.45% normal saline instead of AltaStaph™. The AltaStaph™ or placebo was administered intravenously at a maximum rate of 150 mL/hr. The administration of each dose occurred over about a 4 hour period. Patients were monitored for adverse effects, and in addition, blood cultures, antibody levels, and temperature were monitored. Both AltaStaph™ and the placebo group received conventional therapy, such as antibiotic therapy, to comply with standard of care requirements.

The results, shown below in Tables 4-7, indicate that hyperimmune specific IGIV can be used to effectively treat Staphylococcus aureus infections. The results also show that when hyperimmune specific IGIV is used in combination with conventional antibiotic therapy, patients receiving the hyperimmune specific IGIV enjoy therapeutic medical benefits over those receiving antibiotics alone, such as a shorter time to negative blood cultures and a reduction in the length of hospital stay (a measure of recovery).

AltaStaph™ treated patients had a blood culture negative for S. aureus at an average of 3 days after the first dose of AltaStaph™, while the placebo group did not have a negative blood culture until an average of 4.45 days, as shown in Table 4.

TABLE 4
Days to First Negative S. aureus Blood Culture With No
Recurrence

| | Placebo (N = 11) | AltaStaph ™ (N = 14) |
|---|---|---|
| Mean No. Days | 4.45 | 3.00 |
| Median No. Days | 3.00 | 2.00 |
| Range | 9-12 | 0-7 |
| (Min-Max Days) | | |

Table 5 shows that the average number of days until fever resolution (first temperature less than 38° C. with no subsequent fever) was similar for both groups.

TABLE 5
Days to Resolution of Fever With No Recurrence

| | Placebo (N = 9) | AltaStaph ™ (N = 15) |
|---|---|---|
| Mean No. Days | 2.33 | 2.47 |
| Median No. Days | 1.00 | 1.00 |
| Range (Min-Max | 0-7 | 0-6 |
| Days) | | |

There was a 36% reduction in median time from administration of study drug (AltaStaph™ or placebo) to hospital discharge in the AltaStaph™ treated patients as compared to the placebo treated patients (9 days in the Altastaph group versus 14 days in the placebo group), as shown in Table 6. The reduced hospital stay not only indicates improved treatment, but the reduced hospital stay significantly reduces the cost of treating S. aureus infections.

TABLE 6
Days in Hospital Measured from First Dose of AltaStaph ™

| | | | P Value |
|---|---|---|---|
| | AltaStaph ™ | Placebo | Between |
| | (N = 21) | (N = 18) | Groups |
| Mean Days To | 13.8 (11.6) | 16.2 (12.1) | |
| Discharge (SD) | | | |
| Median | 9 | 14 | 0.0328 |
| Minimum-Maximum | 2-41 | 3-53 | |

AltaStaph™ subjects had a survival rate similar to the placebo group, as shown in Table 7.

TABLE 7
Mortality

| | AltaStaph ™ | Placebo | Total |
|---|---|---|---|
| | (N = 21) | (N = 18) | (N-39) |
| Survived | 16 (76.2%) | 16 (88.9%) | 32 (82.1%) |
| Died | 5 (23.8%) | 2 (11.1%) | 7 (17.9%) |
| Total | 21 | 18 | 39 |

### SUMMARY OF RESULTS

The above-described results are from a clinical trial using Altastaph™ (Staphylococcus aureus Immune Globulin Intravenous (Human)) to treat adult in-hospital patients with persistent Staphylococcus aureus (S. aureus) bloodstream infections (bacteremia). In the study, there was a 36% reduction in median time from administration of the study drug to hospital discharge in the Altastaph™-treated patients as compared to the placebo-treated patients (nine days in the Altastaph™ group versus 14 days in the placebo group). This substantial reduction in the length of hospital stay for the Altastaph™-treated group indicates that S. aureus antibodies provided by Altastaph™ are associated with considerable medical benefit in the treatment of persistent S. aureus infections. The study showed meaningful results in the treatment of patients with a staph infection, and in the treatment of patients with existing serious infections in particular.

The trial was a well-designed clinical study and demonstrated a therapeutic benefit from an antibody therapy in patients with serious infection. These results indicate that the invention will provide a method that will significantly reduce the high costs and serious complications associated with lengthy hospital stays due to S. aureus bacterial infections, because patients treated effectively in accordance with the invention could go home sooner, greatly reducing an increasing burden on the healthcare system.

A more complete analysis of patient data from the same clinical study was conducted. For this analysis, the “median time to clearance of S. aureus bacteremia” and “median time to durable resolution of fever” were determined as time-to-event variables that were described by Kaplan-Meier curves and compared by log-rank or Gehan-Wilcoxon tests. Recurrence of S. aureus bacteremia was examined using a Chi-squared test, and time to recurrence was examined using a Cox model as above. The results are set forth below:

| | Placebo | Altastaph ™ | P |
|---|---|---|---|
| Median time to | 2 days | 1 day | 0.58 |
| clearance of S. aureus | (range: 0-7 days) | (range: 0-6 days) | |
| bacteremia | | | |
| Median time to | 7 days | 2 days | 0.09 |
| durable resolution | | | |
| of fever | | | |
| Median Time To | 14 days | 9 days | 0.03 |
| Hospital Discharge | (range: 3-53 days) | (range: 2-41 days) | |

The following information also was determined:

| | Placebo | Altastaph ™ |
|---|---|---|
| Number of patients | 18 | 21 |
| Number of males | 10 (57%) | 9 (43%) |
| Race: | | |
| White | 9 (50%) | 10 (48%) |
| Black | 5 (28%) | 7 (33%) |
| Hispanic | 3 (17%) | 2 (10%) |
| Other | 1 (6%) | 2 (10%) |
| Mean Weight (kg) +/− SD | 76 +/− 17 | 79 +/− 7.9 |
| Mean APACHE II Score +/− SD | 9.2 +/− 5.2 | 11.7 +/− 7.9) |
| Suspected Source of | | |
| S. aureus bacteremia: | | |
| Bone/joint infection | 4 (22%) | 5 (24%) |
| Catheter-related | 5 (28%) | 2 (10%) |
| IVDU | 2 (11%) | 0 (0%) |
| Endocarditis | 1 (6%) | 2 (10%) |
| Hemodialysis access | 2 (11%) | 5 (24%) |
| Other | 4 (22%) | 2 (10%) |
| Unknown | 0 | 5 (24%) |
| S. aureus serotype: | | |
| Type 5 | 8 | 8 |
| Type 8 | 8 | 5 |
| Type 336 | 5 | 9 |
| Not determined | | 1 |
| Mean Antibody Level | | |
| Day 2 (μg/ml) (95% CI) | | |
| Type 5 | 6.8 (4.8-9.7) | 550 (418-724) |
| Type 8 | 11.9 (9.6-20.6) | 419 (341-515) |
| Mean Antibody Level | | |
| Day 42 (μg/ml) (95% CI) | | |
| Type 5 | 18.6 (7.7-44.8) | 111 (62-197) |
| Type 8 | 20.5 (9.3-45) | 75 (47-120) |

These data show that the method of the present invention provided the patients with high levels of opsonizing antibodies that were effective to treat bacteremia. The efficacy of the method is reflected in a number of different parameters, including the shorter time to clearance of bacteremia, the shorter time to durable resolution of fever, and shorter hospital stays.

### Example 3

### Production of Monoclonal Antibodies to Staphylococcus aureus 336

### A. Immunized Splenocytes Production

A group of 3 BALB/c female mice were immunized with Staphylococcus aureus 336 polysaccharide antigen (either the native, O-acetylated form or a modified, de-O-acetylated form) conjugated to recombinant Exoprotein A (S. aureus 336-rEPA) in combination with Freund's adjuvants. Splenocytes were harvested as a pool from the mice that were administered 3 immunizations at 2-week intervals with test bleeds performed on alternate weeks for serum antibody titers. Splenocytes were prepared as 3 aliquots that were either used immediately in fusion experiments or stored in liquid nitrogen for use in future fusions.

### B. Hybridoma production

Fusion experiments were performed according to the procedure of Stewart & Fuller, J. Immunol. Methods 123: 45-53 (1989). Supernatants from wells with growing hybrids were screened by enzyme-linked immunosorbent assay (ELISA) for monoclonal antibody (MAb) secretors on 96-well ELISA plates coated with S. aureus 336 polysaccharide. ELISA positive cultures were cloned by limiting dilutions, resulting in hybridomas established from single colonies after 2 serial cloning experiments.

### C. Characterization of 336 Monoclonal Antibodies

Each anti-S. aureus 336 MAb reacts strongly to the S. aureus 336 polysaccharide in ELISA and double immunodifusion assays. The MAbs are non-reactive to S. aureus type-5 and type-8 capsular polysaccharides.

### Example 4

### Efficacy of Passive Immunization with Monoclonal Antibodies to Staphylococcus aureus 336

In functional assays, the 336 MAbs are highly effective (in the presence of complement) in promoting the in vitro opsonophagocytosis of S. aureus 336 bacteria with polymorphonuclear cells from human peripheral blood and with HL-60 cells induced with DMSO to differentiate predominantly to cells with metamyelocytic- and neutrophilic- bands. Each MAb also is highly effective in the evaluation of S. aureus isolates to eliminate Type-5 and Type-8 serotypes and confirm 336-specific serotypes. The 336 MAbs also have been shown to be highly effective in promoting survival of mice challenged with lethal doses of S. aureus 336 bacteria after passive immunizations.

Mice were immunized subcutaneously with 200 μL of a monoclonal antibody preparation comprising monoclonal antibodies against S. aureus 336 or E. coli (as a control) (total IgG=500 μg). Mice were challenged with lethal doses of an S. aureus preparation (500 μL at 2.5×10⁵ CFU/500 μL in 5% hog mucin) administered intraperitoneally, and monitored for survival. The following survival results were obtained:

| | | | | | Survival |
|---|---|---|---|---|---|
| | 16 hrs | 24 hrs | 41 hrs | 168 hrs | Rate |
| 1 X PBS | 0/10 | — | — | — | 0% |
| (control) | | | | | |
| S. aureus | 10/10 | 10/10 | 10/10 | 10/10 | 100% |
| mAB 336-119 | | | | | |
| S. aureus mAB | 10/10 | 10/10 | 10/10 | 10/10 | 100% |
| 336-560 | | | | | |
| E. coli mAb | 2/10 | 2/10 | 2/10 | 2/10 | 20% |
| 400 | | | | | |

These results show that the S. aureus 336 monoclonal antibodies achieved 100% protection against the lethal challenge.

### Example 5

### Efficacy of Passive Immunization with Monoclonal Antibodies to Staphylococcus aureus Type 5

Mice were immunized intraperitoneally with a monoclonal antibody preparation comprising one of five monoclonal antibodies against S. aureus Type 5 antigen, a combination of all five Type 5 monoclonal antibodies, or S. aureus Type 5 IGIV. Each mouse received 200 μg antibody or IGIV. Mice were challenged with lethal doses of an S. aureus preparation (5×10⁵ CFU in 5% hog mucin) administered intraperitoneally, and monitored for survival.

The following S. aureus Type 5 monoclonal antibodies were used:

| | Specificity |
|---|---|
| mAb 28D12 | O-acetylated form |
| mAb 053 | O-acetylated + de-O-acetylated forms |
| mAb 529 | de-O-acetylated form |
| mAb 294 | O-acetylated form |
| mAb 072 | O-acetylated form |

The results demonstrated that each monoclonal antibody achieved significant protection, and that the combination preparation achieved a level of protection equivalent to that achieved by IGIV in this study, as reflected in the following survival data:

| | 16 hrs | 18 hrs | 22 hrs | 25 hrs | 39 hrs | 42 hrs | 46 hrs | 6 day | 7 day |
|---|---|---|---|---|---|---|---|---|---|
| mAb | 13/15 | 13/15 | 10/15 | 10/15 | 10/15 | 10/15 | 10/15 | 10/15 | 10/15 |
| 28D12 | | | | | | | | | |
| mAb | 13/15 | 12/15 | 11/15 | 11/15 | 11/15 | 11/15 | 11/15 | 11/15 | 11/15 |
| 053 | | | | | | | | | |
| mAb | 14/15 | 12/15 | 12/15 | 12/15 | 12/15 | 12/15 | 12/15 | 12/15 | 12/15 |
| 529 | | | | | | | | | |
| mAb | 14/15 | 14/15 | 13/15 | 13/15 | 13/15 | 13/15 | 13/15 | 13/15 | 13/15 |
| 294 | | | | | | | | | |
| mAb | 15/15 | 15/15 | 15/15 | 15/15 | 14/15 | 14/15 | 14/15 | 14/15 | 14/15 |
| 072 | | | | | | | | | |
| mAb | 14/15 | 14/15 | 14/15 | 14/15 | 14/15 | 14/15 | 14/15 | 13/15 | 13/15 |
| comb. | | | | | | | | | |
| T5 IGIV | 14/15 | 14/15 | 14/15 | 14/15 | 14/15 | 14/15 | 14/15 | 14/15 | 14/15 |
| PBS | 14/15 | 9/15 | 8/15 | 5/15 | 5/15 | 5/15 | 5/15 | 4/15 | 4/15 |
| (control) | | | | | | | | | |

### Example 6

### Dose Response Study of S. aureus Type 5 IGIV & Monoclonal Antibodies

Mice were immunized intraperitoneally with varying doses of S. aureus Type 5 IVIG, varying doses of one of two Type 5 monoclonal antibody preparations (O-acetylated and de-O-acetylated), or a Type 8 monoclonal antibody preparation. Mice were challenged with lethal doses of an S. aureus preparation (5×10⁵ CFU in 5% hog mucin) administered intraperitoneally, and monitored for survival.

| | 16 hrs | 18 hrs | 22 hrs | 25 hrs | 39 hrs | 42 hrs | 46 hrs | 5 day |
|---|---|---|---|---|---|---|---|---|
| 400 μg | 100% | 100% | 100% | 100% | 100% | 100% | 100% | 100% |
| T5 IVIG | | | | | | | | |
| 200 μg | 93% | 93% | 93% | 93% | 93% | 93% | 93% | 93% |
| T5 IVIG | | | | | | | | |
| 100 μg | 100% | 100% | 100% | 100% | 100% | 93% | 93% | 93% |
| T5 IVIG | | | | | | | | |
| 50 μg T5 | 93% | 93% | 93% | 93% | 93% | 93% | 93% | 93% |
| IVIG | | | | | | | | |
| 200 μg | 86.6% | 80% | 80% | 80% | 80% | 80% | 80% | 80% |
| mAb 072 | | | | | | | | |
| 100 μg | 100% | 93% | 93% | 93% | 93% | 93% | 93% | 93% |
| mAb 072 | | | | | | | | |
| 50 μg | 86.6% | 86.6% | 80% | 80% | 73% | 73% | 73% | 66% |
| mAb 072 | | | | | | | | |
| 200 μg | 100% | 86.6% | 80% | 80% | 73% | 73% | 73% | 66% |
| mAb 053 | | | | | | | | |
| 100 μg | 93% | 93% | 93% | 93% | 86% | 86% | 86% | 86% |
| mAb 053 | | | | | | | | |
| 50 μg | 73% | 66% | 66% | 66% | 53% | 53% | 53% | 40% |
| mAb | | | | | | | | |
| 0053 | | | | | | | | |
| 200 μg | 86.6% | 86.6% | 73% | 53% | 47% | 47% | 40% | 40% |
| T8 mAb | | | | | | | | |
| PBS | 60% | 53% | 40% | 27% | 27% | 27% | 27% | 27% |
| (control) | | | | | | | | |

These results show that each monoclonal antibody achieved significant protection.

While preferred embodiments have been illustrated and described, it should be understood that changes and modifications can be made in accordance with ordinary skill in the art without departing from the invention in its broader aspects as defined herein.

The contents of each document cited herein is expressly incorporated herein by reference in its entirety.

What is claimed is:

1. A method of treating S. aureus bacteremia comprising:
administering to a patient suffering from S. aureus bacteremia an effective amount of an antibody composition comprising antibodies specific for one or more antigens of S. aureus.

2. The method of claim 1, wherein the antibody composition is an IGIV composition.

3. The method of claim 2, wherein the antibody composition is a hyperimmune specific IGIV composition.

4. The method of claim 1, wherein the antibody composition comprises recombinant antibodies.

5. The method of claim 1, wherein the antibody composition comprises monoclonal antibodies.

6. The method of claim 1, wherein the antibody composition comprises antibodies specific to one or more capsular polysaccharide antigens of Staphylococcus aureus.

7. The method of claim 6, wherein the antibody composition comprises antibodies specific to one or more antigens selected from the group consisting of the Type 5 antigen, the Type 8 antigen, and the 336 antigen.

8. The method of claim 7, wherein the antibody composition comprises antibodies specific to the Type 5 antigen and the Type 8 antigen.

9. The method of claim 7 wherein the antibody composition comprises antibodies specific to the 336 antigen.

10. The method of claim 7, wherein the antibody composition comprises antibodies specific to the Type 5 antigen, the Type 8 antigen, and the 336 antigen.

11. The method of claim 1, wherein the bacteremia is characterized by a persistent fever.

12. The method of claim 1, wherein the bacteremia is caused by antibiotic resistant Staphylococcus.

13. The method of claim 12, wherein the Staphylococcus is resistant to methicillin.

14. The method of claim 12, wherein the Staphylococcus is resistant to vancomycin.

15. The method of claim 1, wherein the patient is immunocompromised.

16. The method of claim 1, wherein the patient is allergic to at least one antibiotic used to treat Staphylococcus.

17. The method of claim 1, further comprising an additional therapy against Staphylococcus infection.

18. The method of claim 17, wherein the additional therapy comprises the administration of one or more antibiotics.

19. The method of claim 18, wherein the additional therapy comprises the administration of one or antimicrobial agents.

20. The method of claim 19, wherein the additional therapy comprises the administration of lysostaphin.

21. The method of claim 1, wherein the antibody composition comprises an immunostimulatory compound.

22. The method of claim 22, wherein the immunostimulatory compound is selected from the group consisting of B-glucans and GM-CSF.

23. The method of claim 1, wherein the antibodies comprise antibodies specific for the native form of one or more antigens of S. aureus.

24. The method of claim 1, wherein the antibodies comprise antibodies specific for a modified form of one or more antigens of S. aureus.

25. The method of claim 24, wherein the antibodies comprise antibodies specific for a de-O-acetylated form of an S. aureus Type 5 antigen or a de-O-acetylated form of an S. aureus Type 8 antigen.

26. A method of preventing S. aureus bacteremia comprising:
administering to a patient at risk for developing S. aureus bacteremia an effective amount of an antibody composition comprising antibodies specific for one or more antigens of S. aureus.
